(19) United States
(12) Patent Application Publication
(10) Pub. No.: US 2013/0195827 A1
(43) Pub. Date: Aug. 1, 2013

(54) NUTRIGENOMIC METHODS TO OVERCOME CARBOHYDRATE BINGEING AND OVEREATING

(71) Applicants: Kenneth Blum, San Diego, CA (US); B. William Downs, Lederach, PA (US); Roger L. Waite, San Diego, CA (US); William J. Heaney, Huntington Beach, CA (US)

(72) Inventors: Kenneth Blum, San Diego, CA (US); B. William Downs, Lederach, PA (US); Roger L. Waite, San Diego, CA (US); William J. Heaney, Huntington Beach, CA (US)

(21) Appl. No.: 13/629,581

(22) Filed: Sep. 27, 2012

Related U.S. Application Data
(60) Provisional application No. 61/539,976, filed on Sep. 27, 2011.

(51) Int. Cl. A61K 31/198; A61K 36/45; A61K 36/185; A61K 31/555; A61K 31/435; A61K 31/51; A61K 31/205; A61K 31/194; A61K 36/27; A61K 33/06; A61K 36/41; A61K 31/4415

(52) U.S. Cl. CPC A61K 31/198; A61K 36/41; A61K 36/45; A61K 36/185; A61K 31/555; A61K 31/4415; A61K 31/51; A61K 31/205; A61K 31/194; A61K 36/27; A61K 33/06; A61K 31/435
USPC 424/94.1; 514/348

(57) ABSTRACT

This invention concerns Reward Deficiency Syndrome (RDS) and obesity, and the role of catecholaminergic pathways in aberrant substance-seeking behavior, in particular cravings for carbohydrates. Also described are new nutrigenomic formulas having unique combinations of ingredients having a generalized anti-craving effect are, which can inhibit carbohydrate bingeing, inducing significant healthy fat loss and relapse prevention, as well genetic testing for certain polymorphisms correlated with RDS behaviors.

### BACKGROUND OF INVENTION

It is well-known that there is an obesity epidemic worldwide. To alter this trend, new strategies and programs for weight maintenance as well as weight reduction must become a high public health priority. This invention provides a patentable new approach.

“Weight loss,” “weight gain”, and “weight management” are the most common terms used to express changes in body composition, particularly regarding fat mass. However, as shown herein, this focus on “weight” as an accurate measuring criterion poses a contradiction to the natural sequence of processes in recompositional metabolism, creates inappropriate expectations, and does not provide a correct or accurate perspective for evaluating healthy changes in body composition, as fat is the lightest of pertinent macromolecules. More importantly, fat is usually the last to go in the body recomposition process, therefore, creating short-term expectations is erroneous.

Fat metabolism is influenced by many factors, from genetics to lifestyle and the efficiency of energy metabolism. Existing “weight loss” tactics for the most part have failed to provide successful means to achieve sustainable healthy body composition and improve healthy fat loss. Commercialized “weight loss” programs, even those that are medically supervised, do not consider the “bi-phasic” nature of genetically regulated set-point “defense response” mechanisms that mandate preservation of body fat stores against famine and survival threats simulated by aggressive weight loss tactics during phase 1. Further, existing tactics erroneously emphasize caloric intake to the exclusion of considering nutrient quality and density of those calories, a factor far more important to metabolic competence than calories alone.

This invention concerns a new body recomposition and healthy body mass management technology. Patentable formulas and methods to safely and naturally induce effective body recomposition and achieve healthy body mass management objectives are described. This invention contrasts with existing tactics to manipulate body composition, in that it is based on the fact that sufficient nutrition (as opposed to just calories) is required to fund a wide range of factors involved in achieving healthy and efficient metabolic function. This invention synergistically combines nutraceutical ingredients necessary to simultaneously address symbiotic mechanisms that promote healthy metabolism in the energy management system, stress and inflammation management system, the pleasure/food craving management system (controlled by the brain), the immune management system, and the neuroendocrine system. Importantly, these five systems are homeostatic and intimately interactive and interdependent in ensuring optimal metabolic function. This instant invention optimizes genetically programmed energy expenditure and storage functions, without inducing “Yo Yo” rebound weight gain consequences. In contrast to conventional short term expectations, “weight loss” might not be expected since the need to improve the health of the cellular energy-producing apparatus might first result in increased muscle density and weight “gain” that is needed to promote healthy and permissible fat oxidation and loss. In fact, a more normal sequence of events can include initial water weight loss, increased muscle density and weight (since muscle is more dense than fat and/or water) followed by permissible fat loss, which can take many months to achieve. Such a sequence could and has contributed to disappointment with short term “weight loss” results and abandonment of more intelligent programs that would lead to sustainable fat loss in the healthy body recomposition dynamic.

Various minerals have been shown to be important in funding events leading up to and promoting healthy carbohydrate metabolism, insulin function, energy production, fat oxidation, serotonin release and availability in the brain, blood lipid metabolism, and improving the success of fat loss and body composition management efforts (see FIG. 1).

Based on the premise of this novel nutraceutical technology presented herein provides ample evidence that the term “weight loss” is a misnomer. This term “weight loss” (or any terms using the “weight” language reference) appearing in quotations is deliberately misused herein to emphasize the point of how conventional tactics (and language) contribute to erroneous, but unquestionably accepted, dogma. Current “weight loss” tactics, for the most part, are based on inducing calorie intake deprivation and artificial stimulation, deprivation, and/or inhibition the body's genetically programmed energy expenditure, storage, regulatory, and management processes. These types of tactics include, but are not limited to:

Central Nervous System Stimulants (CNSS) that artificially stimulate the rate of calorie burning (Basal Metabolic Rate [BMR]).

### Appetite Suppressants

### Fat Blockers

### Starch Blockers

### Diuretics (Water Pills)

### Low Calorie Diets

### Low Food Diets

Meal Replacement Programs (Diet Shakes, bars, etc.)

### High Protein Diets

### High Carbohydrate Diets

### Low/No Carbohydrate Diets

### Low Fat Diets

### Pre-Meal Fiber/Water “Fill-You-Up” Programs

Fruit and Fruit juice “Rapid “weight loss”” Programs
Over Night “weight loss” Programs

### Vegetable Soup Diet Programs

### Liposuction

### Radical Digestive Tract Surgeries

### Acupuncture

### Laxatives

### Hypnosis

Many of these tactics are used individually or in combination to achieve rapid “weight loss” results. As stated, the primary goal of these tactics is “weight loss” and/or image enhancement. These objectives are usually pursued without regard for or knowledge of the impact on health, the body's natural genetically mandated homeostatic response to such tactics, or the fact that depriving the body of resources essential to maintain health is counterproductive. Essentially, these types of tactics simulate the circumstances of a famine and induce genetically programmed energy conservation responses. In addition, at some point in the energy conservation sequela, increased appetite can result. Alarmingly, many of these tactics are approved, administered, and/or supervised by medical or health professionals. While initially appearing to promote “weight loss” (phase 1), such tactics are destined to fail as gene-induced recalibration of energy management and storage instructions homeostatically adjusts to the artificially imposed influence of such tactics, generally by lowering the basal metabolic rate, increasing energy storage requirements and promoting increased fat retention (phase 2). Chronic and repeated attempts to lose weight with such tactics are referred to as the yo-yo weight gain rebound effect. This phenomenon is responsible for ever-increasing frustration, anxiety and a sense of helplessness caused by the out-of-control “weight loss”/gain juggernaut.

Ultimately, obesity is an energy-balance and nutrient deficiency-induced famine disorder characterized by a survival gene induced increase in fat storage, lowering of the Basal Metabolic Rate (to conserve energy) and increase in appetite. Following circumstances when a simulated famine is induced, certain genes, programmed to resist loss of body fat, prevail. This programmed genetic predisposition is responsible for down-regulating the resting metabolic rate (RMR) in response to dietary and caloric restriction, which is significantly disrupted following rapid “weight loss” regimens, like those tactics indicated above. Over-consumption of food, especially nutritionally deficient high calorie food (excess energy intake), is a normal consequence contributing to weight gain and obesity.

A resistance to the hormone leptin also characterizes common obesity. Insulin has been shown to increase leptin secretion by 25%. Ample evidence demonstrates that insulin resistance is also a primary contributor to obesity, suggesting that insulin resistance induced hyperinsulinemia can provoke leptin resistant hyperleptinemia with a consequential increase in fat synthesis and storage in adipocytes, characteristic sequela of Syndrome X or Metabolic Syndrome. Further, adipocytes from fatter animals secrete more leptin and a correlation between intracellular ATP concentration and the rate of leptin secretion appears to exist. As such, leptin concentration correlates positively with percent body fat. A low resting metabolic rate (RMR) for a given body size and composition, a low rate of fat oxidation, and low levels of physical activity are risk factors for weight gain and common traits of obese individuals. It has been shown that a decrease in body weight as fat mass and fat free mass is accompanied by a greater decrease in resting energy expenditure and fat oxidation.

### SUMMARY OF INVENTION

Effective fat loss and body recomposition strategies addressing the energy management pathways should simultaneously improve insulin, serotonin, and fat oxidation metabolism; potentiate a healthy increase in RMR and energy expenditure; and blunt excessive appetite cravings, given proper adequate nutrient and energy intake. The technology of the present invention replenishes the nutritional needs of at least five important systems, which are essential to healthy weight management:

1. The biochemical mechanisms involved in nutrition and energy management regulating intake, expenditure and storage controls and feedback;

2. Attenuation of the effects of chronic stress and inflammation (which overburden the endocrine system and can cause, for example, excessive cortisol production), reducing fat storage;

3. The pleasure seeking needs and reward circuitry of the brain, influencing psychological and emotional need-induced food cravings;

4. Promotion and support of healthy immune system function (involved in catalyzing survival response to metabolic threats); and

5. Supporting and maintaining optimal health of the neuroendrocrine system through which the majority of metabolic signaling is processed. Nutritional and gene expression deficiencies in the reward neurochemical pathway limit the brain's reward resources (specific neurotransmitters) and are responsible for a condition called “Reward Deficiency Syndrome” (RDS), which causes excessive cravings.

RDS results from a dysfunction in the Brain Reward Cascade, which directly links abnormal craving behavior with a defect in the DRD₂ Dopamine Receptor Gene as well as other dopaminergic genes (D1, D3, D4, D5). Dopamine is a very powerful neurotransmitter in the brain, which controls feelings of well-being. This sense of well-being is produced through the interaction of dopamine and neurotransmitters such as serotonin, opioids, and other powerful brain chemicals. Low serotonin levels are associated with depression. High levels of the opioids (the brain's opium) are associated with a sense of well-being. The complex interactions of these powerful neurotransmitters, ultimately regulating the Dopaminergic Activity in the Reward Center of the Brain, have been termed “The Brain Reward Cascade” (see FIG. 2).

In individuals possessing an abnormality in the DRD2 Dopamine Receptor Gene, the brain lacks enough Dopamine receptor sites to use the normal amount of Dopamine in the Reward Center of the brain and thus reduces the function of Dopamine in this area of the brain. Individuals possessing the variant in the Dopamine Receptor Gene tend to be serious cocaine abusers, may have unhealthy appetites that can lead to obesity or overeating.

On the other extreme, these individuals can be anorexic with extremely low caloric intake, have levels of stress over an extended time period and their addictive brains lead to high-generalized craving behavior. In essence they seek substances including alcohol, cocaine, nicotine, and/or glucose (substances known to cause preferential release of dopamine at the nucleus accumbens) to activate dopaminergic pathways as a self-healing process to offset their low D2 receptors caused by genetic antecedents known as the dopamine D2 receptor gene Taq1 A1 allele.

The overall effect is inadequate Dopaminergic Activity in the Reward Center of the Brain. This defect drives individuals to engage in activities, which will increase brain Dopamine function. Consuming large quantities of alcohol or carbohydrates (carbohydrate bingeing) stimulate the brain's production of and utilization of Dopamine. So too does the intake of crack/cocaine and the abuse of nicotine. Also, it has been found that the genetic abnormality is associated with aggressive behavior, which also stimulates the brain's use of Dopamine.

RDS involves a form of sensory deprivation of the brain's reward or pleasure mechanisms. RDS can be manifested in relatively mild or severe forms that follow as a consequence of an individual's biochemical inability to derive reward from ordinary, everyday activities. The inventors believe that they have discovered at least one genetic aberration that leads to an alteration in the reward pathways of the brain. This aberration is a variant form of the gene for the dopamine D₂ receptor, called the A1 allele. This genetic variant also is associated with a spectrum of impulsive, compulsive, and addictive behaviors. The RDS concept unites those disorders to explain how simple genetic anomalies give rise to complex aberrant behavior (see FIG. 3).

This specification share the scientific evidence explaining why people overeat and become overweight in a society where “thin is in”. Initially, it is helpful to consider the relationship between eating behavior and “brain chemistry”, and the interaction of both genetics and environmental elements.

### BRIEF DESCRIPTION OF THE DRAWINGS

FIG. 1 is a diagram showing the role of the nutrigenomics of Neuradaptogen amino acid therapy (NAAT™) as an anti-obesity complex.

FIG. 2 is a diagram showing the anatomy of the brain's reward system.

FIG. 3 is a table re Reward Deficiency Syndrome (RDS) that categorizes certain RDS-associated behaviors.

FIG. 4 is a diagram showing the relationship of various neurotransmitters in the Reward Cascade

FIG. 5 illustrates a “happiness gene map” that shows five polymorphic genes (circled) used as obesity nutrigenomic therapeutic targets.

FIGS. 6 and 7 are bar graphs that plot the effects of the KB220 formulation on weight and body mass index (BMI).

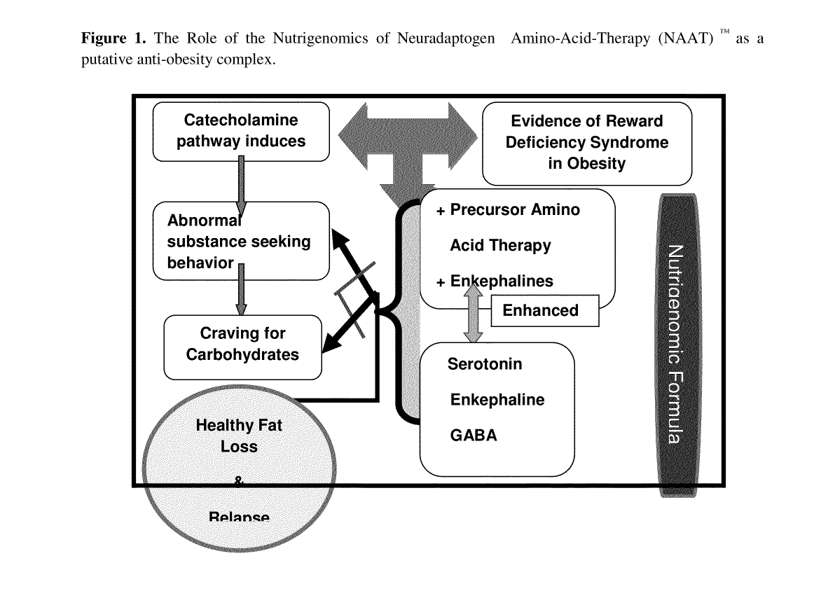

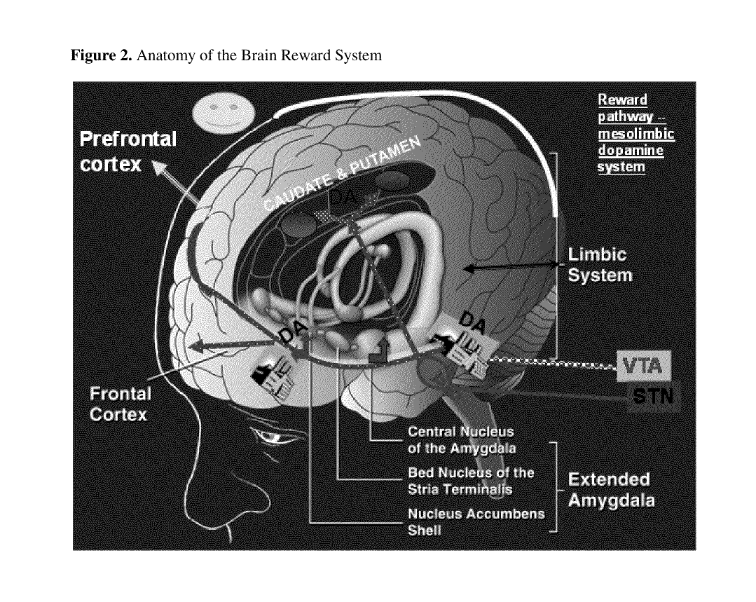

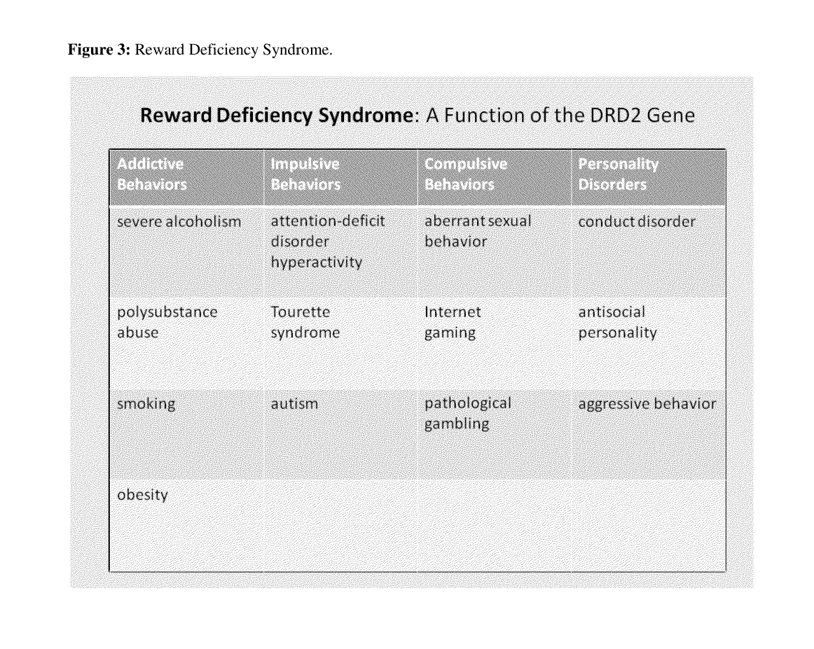

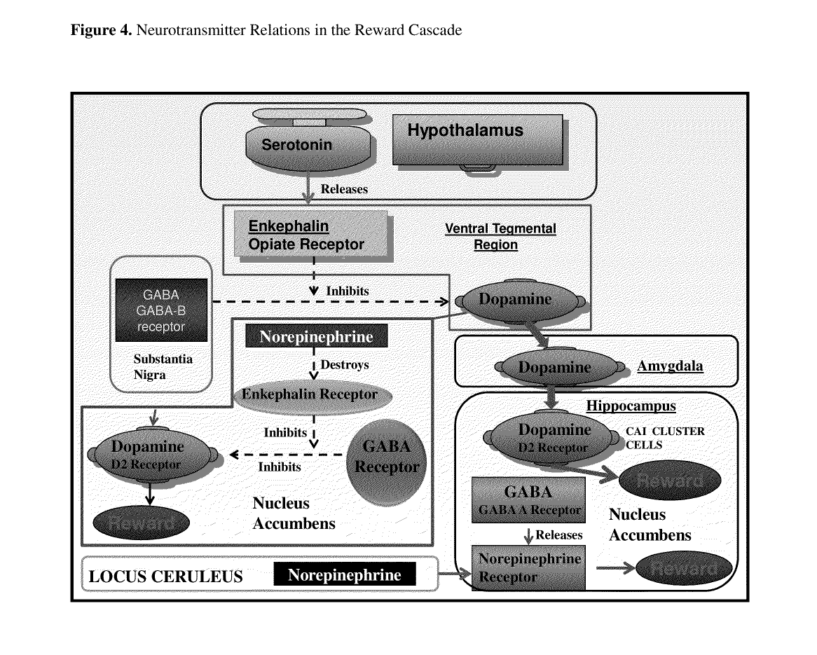

### WEIGHT GAIN: A PREHISTORIC LOOK

Unlike today, in prehistoric times hunter-gatherers did not continually have a plentiful food supply. For example, when pristine sources of nutrient-rich berries and roots were in season and when wild animals were not hibernating, they ate well enough to allow their bodies to produce fat from excess food intake. However, when such foods were less plentiful or not available, they relied on stored fat to compensate for inadequate food intake in order to survive.

To help understand the importance of human weight gain, two biological functions assisted human ancestors as they struggled to survive this perpetual cycle, colloquially known as “feast” and “famine”. When there is an abundant supply of high quality food, our bodies efficiently produce and store fat, and during times when there is a lack of food, metabolism slows. Today scientists believe that abundant food induced efficient fat storage, but when one has less than biologically sufficient, fat metabolism slows to adjust to the smaller quantities and metabolic rates are adapted to food intake. Those who survived are believed to have evolved genetics coding for efficient fat production and storage genes, while those who lacked these genes perished. This allowed the survivors to pass their “thrifty” genes on to future generations, and ultimately to modern humans. Thus, over time the genes involved in fat production and storage evolved to allow us to store energy from nutrient-deficient concentrated sugars, processed carbohydrates, and adulterated fats, which in turn allows humans to survive the “famine” that chronic intake of these types of low quality “foods” simulate.

Today's obesity epidemic contributes to an estimated 300,000 premature deaths annually in the United States, and the number of obese in the United States is doubling every five years. In fact, obesity is a contributing risk factor to four of the seven leading causes of death. The Center for Disease Control has stated that obesity is the number one health risk, greater than a lifetime of smoking, drinking, and poverty.

Of course, in the modern world today, most Americans do not struggle through periods with very little food. Instead, we live in an environment perpetually filled with calorie-rich, nutrient-deficient foods, the result of which is that our bodies operate in a genetically preprogrammed fat storage mode; however, if a “low-fat diet” is then pursued, our bodies, responding again in a genetically preprogrammed way, signal urge to “eat”. Thus, in all of us, there is a rebound effect, which leads to quickly regaining any lost weight in preparation for evolutionarily anticipated next food shortage, as invariably happened to ancestral humans. As put in 1996, “[The] modern western lifestyle appears to provide the social and environmental conditions that favor maximum expression of underlying individual genetic differences in susceptibility to becoming overweight.”

Thus, it is important to understand that humans that, with regard to metabolic effects, the body's instinct is to prepare for and defend against famine, which is problematic in a modern society that has largely eliminated hunger and famine. There is, though, an even more important facet to the genetic propensity to gain excess weight, and it does not reside in genes that control fat storage and/or resting metabolic rates. Instead, it is in the genes that control the desire “to Binge or not to binge”, namely, the genes termed “reward genes”.

### PREFERRED EMBODIMENT

Since there is a strong link between sugar craving (ultimately leading to obesity) and hypodopaminergic activity in the brain most likely due to a number of polymorphic genes, the invention concerns patentable combinations of natural ingredients to promote well-being, stress reduction, enhanced energy, increased BMR, enhanced executive functioning, reduced sugar craving, reduced need to binge, increased insulin sensitivity, enhanced immune response, neurotransmitter balance, increased dopaminergic function, relapse prevention, fat loss, weight loss, BMI reduction, and, most importantly, prevention regain of already lost weight from other short term programs. In particular, the formulations and methods of the invention rely on a composition useful to treat RDS and at least one other active ingredient or agent.

Regarding compositions useful to treat RDS, preferred examples are provided in U.S. Pat. No. 6,132,724, which is hereby incorporated by reference in its entirety for any and all purposes. Preferred examples of such compositions include: (a) at least one substance that inhibits the enzymatic destruction of an opioid (opiate) neuropeptide (preferably an amino acid, peptide, or structural analogue or derivative thereof); (b) a neurotransmitter synthesis-promoting amount of at least one neurotransmitter precursor, preferably a dopamine precursor such as L-Tyr, L-Phe, or L-Dopa, a serotonin precursor (e.g., L-Trp, and 5-hydroxytryptophan), and/or a gamma amino butyric acid (GABA) precursor, for example, L-glutamine, 1-glutamic acid, and L-glutamate (or GABA itself); (c) a tryptophan concentration-enhancing amount of any chromium salt (for example chromium picolinate or chromium nicotinate); and (d) a neurotransmitter synthesis-promoting amount of at least one neurotransmitter synthesis promoting substance or catabolic inhibitor selected from the group consisting of rhodiola and huperzine. A metalloglucoside may also be included. The preferred dosages, dosage forms, and routes and methods of administrations for such compositions include those as are described in the '724 patent.

As for the other active ingredient or agent, it is preferably a nutraceutical or plant extract, or purified form or analogue or derivative of a chemical that naturally occurs in a plant. Preferred examples of such other ingredients include one or more of the following:

Rhododendron. Rhododendron is an Asian plant that has important fat-reducing benefits. Studies in conjunction with Rhodiola 200 mg in combination with Rhododendron (100 mg) in double blind studies show significant human “weight loss”.
(−)-Mineral salts of (−)Hyroxycitric acid (HCAMin) cause fat loss in humans without stimulating the central nervous system. HCAMin is derived from the fruit rinds of Garcinia cambogia, which exhibits a distinctive sour taste and has been used for culinary purposes on southern Asia. HCA is then reacted with mineral salts, usually potassium, calcium and more recently magnesium. HCAMin is a competitive inhibitor of ATP-citrate lyase, an extra-mitochondrial enzyme involved in the initial steps of de novo lipogenesis. Consequently, HCA reduces the transformation of citrate into acetyl coenzyme A, a step necessary for the formation of fatty acids in the liver. In addition, there is increased production of liver glycogen in the presence of HCAMin, which may activate glucoreceptors leading to a sensation of fullness and reduced appetite. Dosage is preferably suboptimal, preferably less than 1500 mg/day.
Gymnea Syveste. GS helps to reduce undesirable fat formation by its ability to reduce cravings for sweets and control blood sugar levels. A peptide isolated from Gymnema, gurmarin, has also been shown to block the sweet taste of glucose and sucrose in animals. Gurmarin temporarily binds the sweet and bitter receptors on the tongue, thereby blocking the taste sensation and reducing sweet cravings. Dosages of Gymnema sylvetre of about 10-4,000 mg can be used, with a dose of about 400 mg being preferred (providing 100 mg gymnemic acid).
Carnitine (optional ingredient), promotes fat metabolism
Calcium, which promotes neurotransmitter release.
Passiflora incarnata

Passion flower is a name that has been given to several members of the genus Passiflora. There are more than 40 species in the genus whose origins are in both the tropical and subtropical regions of the western hemisphere. Passion flower was first brought to Europe from Mexico in the sixteenth century by Spanish conquerors. Its main medicinal purpose was that of a calming tea. It is now part of the medicinal herbarium in many countries throughout the world. Passion flower's long history in herbal medicine includes its use as a treatment for colic, diarrhea, dysentery, menstrual pain, skin eruptions, conjunctivitis, hemorrhoids, and myscle spasms.

It is important to properly identify the Passion flower plant. While there are a number of alkaloids that have been sold under the rubric of Passion flower, the most important and consistently effective candidate is Passiflora incarnata. The ethnobotanical database on the U.S. Agricultural Research Service's website lists the total alkaloid content of P. incarnata as 100 to 900 ppm and the total flavinoid content as 1.2-3.9 percent, which has been further tested by others. Twenty-six components fall into two categories: 20 flavonoids (including a cyanogenic glycoside and gynocardine) and 6 alkaloids. Some researchers have ascribed the sedative effects of P. incarnata to indole alkaloids such as Harmane and its relatives, harmaline and harmol. However, others have suggested that P. incarnata's alkaloid content is too small to cause this and other CNS effects and that flavonoids—such as apigenin, luteolin, or their glycosides—are more likely to account for CNS bioactivity. Most recently, scientists have isolated a highly anxiolytic, trisubstituted benzoflavone moiety from a P. incarnata extract. Reportedly, this extract has the ability to restore libido on aging male rats and those who are addicted to tetrahydrocannibinol to restore fertility and libido that has been reduced by alcohol or nicotine use, and to reduce the anxiety arising from alcohol withdrawal. There are also double-blind randomized studies that show that Passiflora extract is as effective substance for the management of generalized anxiety disorder compared to the drug Oxazepam. There is even evidence from a double-blind randomized controlled trial that Passiflora can serve as an effective adjuvant in the management of opiate withdrawal of opiates. In addition, Passiflora has been shown to reduce benzodiazepone dependence in mice. In fact, many pharmacological investigations confirm the sedative effects of Passiflora, especially in the P. incarnata form. In certain preferred formulations of the invention, fragmented or cut, dried aerial parts of P. incarnata (preferably excluding flowers) are used, as separated leaves afford the best CNS results.

As will be appreciated, the amount of a composition according to the invention administered to or taken by a patient or subject is effective for the prevention or treatment of unwanted weight gain associated with metabolic syndrome and/or the attenuation of a number of symptoms, including, but not limited to, enhancement of well-being, stress reduction, enhanced energy, increased BMR, enhanced executive functioning, reduced sugar craving, reduced need to binge, increased insulin sensitivity, enhanced immune response, neurotransmitter balance, increased dopaminergic function, relapse prevention, fat loss, weight loss, BMI reduction, and prevention regain in already lost weight from other short term weight loss programs.

### Example of Particularly Preferred Compositions

The following table lists the ingredients of a particularly preferred formulation according to the invention, termed SEP711C3G, along with dosage ranges and desire therapeutic dosages (where established):

TABLE 1
SEP711C3G

| | Desired Thera- | | |
|---|---|---|---|
| Ingredient | peutic Dose | | Dosage Range |
| DL-Phenylalanine | 2170 | mg | 10 mg to 10.000 mg |
| L-tyrptophan | | | 1 mg to 2000 mg |
| 5-Hydroxytryptaphan | 50 | mg | 1 mg to 500 mg |
| L-Glutamine | 50 | mg | 1 mg to 500 mg |
| L-Tyrosine | 775 | mg | 1 mg to 5000 mg |
| Chromium Polynicotinate | 4 mg (400 mcg | | 10 mcg to 100 mg |
| | elemental Cr) | | |
| Rhodiola | 170 | mg | 5 mg to 200 mg |
| Passion Flower | 300 | mg | 1 mg to 3000 mg |
| (incarnata) | | | |
| Vitamin B6 | 20 | mg | 10 mcg to 500 mg |
| Pyridoxine HCl (10) | | | |
| Pyridoxal 5-Phosphate (10) | | | |
| Vitamin B1 (Thiamine) | 31 | mg | 10 mcg to 1000 mg |
| Carnitine | 250 | mg | 1 mg to 5000 mg |
| Rhododendron | 100-200 | mg | 1 mg to 5000 mg |
| (−)- Mineral salts | 4500 | mg | 10 mg to 10,000 mg |
| of (−)Hyroxycitric | | | |
| acid (HCAMin) | | | |
| Gymnema sylvestre | 25 | mg | 1 mg to 500 mg |
| Calcium | 1200 | mg | 1-3000 mg |
| CogniTrim ™ | (combination of | | |
| | SH1028 &NOPE2G2) | | |
| CurQFen ™ | 100 | mg | 1 mg to 2000 mg |
| PhosphoLean ™ | 100 | mg | 1 mg to 5000 mg |
| SH1028 Choline | 275 | mg | 1 mg to 5000 mg |
| alphoscerate | | | |
| GlucodOX ™ | 70 | mg | 1 mg to 3000 mg |
| Irvingia gabonensis | 300 | mg | 1 mg to 5000 mg |
| seed extract (OB131) | | | |
| Huperzine A | 20 | mg | 100 mcg to 1000 mg |
| (1% concentration) | | | |
| Bauhinia variegata | 100 | mg | 1 mg to 2000 mg |
| extract | | | |
| Niacin | 30 | mg | 1-100 mg |
| Taurine | 500 | mg | 1 mg to 3000 mg |
| Kola Nut | 300 | mg | 1 mg to 3000 mg |
| Zehntose | 200 | mg | 1 mg to 5000 mg |
| Metallosaccharides | | | |
| complex | | | |
| Green Tea extract | 100 | mg | 1 mg to 2000 mg |
| Weight Management | 100 | mg | 1 mg to 5000 mg |
| Enzyme Complex | | | |

Based on consistent positive research outcomes and technology, the following nutrients are scientifically formulated (following meticulous ingredient selections and dosage determinations), have been clinically tested, and have demonstrated profound efficacy at supporting optimal brain health; improving craving management; enhancing energy expenditure, neuroendocrine function, memory, focus, and cognition; immune competence; stress reduction; and body composition and weight management.

Below is described the constituent parts of a composition known as CogniTrim™, which can serve as the other active ingredient in a formulation according to the invention.

CurQFen™—Superior to other curcumin products by up to 125 times, CurQFen is a fully reacted patent pending BR213 Curcuma galactomannosides compound that promotes: cognition, healthy cardiac function, immune competence, and a healthy gut; reduces the need to activate inflammatory cytokines; helps maintain blood sugar and blood lipid levels within the normal range; and slows the absorption of carbohydrates, cholesterol, bile acids, and improves gastric emptying.
PhosphoLean™ NOPE2G2 is a patented, advanced, appetite regulating and weight management compound that is clinically proven to help people control binge eating, and lower depressed feelings, all keys to successful, long-term weight loss. PhosphoLean™ can increase satiety, decrease depressive symptoms, decrease binge-eating severity, and provide favorable changes in insulin resistance and lipids. The EGCG polyphenols in PhosphoLean™ NOPE2G2 act synergistically via sympathetic activation of thermogenesis and increase fat oxidation, thus enhancing the compound's weight management effects. PhosphoLean also significantly improves diet compliance in a group of healthy, overweight or obese subjects.
SH1028 Choline alphoscerate—After consumption, SH1028 Choline alphoscerate is converted to the metabolically active form of choline able to reach cholinergic synaptic endings, thus increasing acetylcholine release. Metabolically active choline prevents fat deposits in the liver and facilitates the movement of fats into the cells. SH1028 promotes significant improvement in cognition, memory, and other neuro-chemical and -psychological cholinergic-dependent structures and functions, such as parasympathetic and sympathetic nervous system functions, neuromuscular junctions, basal forebrain function (considered to be the major cholinergic output of the central nervous system (CNS)), and important for healthy brain stem complexes. In addition, acute supplementation augments growth hormone response to, and peak force production during, resistance exercise.
GlucodOX™ is a nutraceutical ingredient complex comprised of a supercritical Commiphora mukul extract and a medium chain triglyceride (MCT) oil composed of C8 and C10 fatty acids. GlucodOX™ contains guggulsterones (standardized to 2.0% by HPLC analysis), which have been linked to several mechanisms that support lipid metabolism, glucose metabolism and cellular energy. GlucodOX™ properties are enhanced by MCTs, which can gain rapid access to the mitochondria (energy producing organelle in cells). Given their high energy density, rapid rate of absorption, and quick metabolic conversion into cellular energy, MCTs can be used for fueling physical exertion.

The GU-MCT810 complex in GlucodOX can support:
glucose metabolism
cholesterol levels already within the normal range and blood lipid metabolism
mitochondrial biogenesis (supports the creation of new energy-producing mitochondria)
primary energy production
   SEBTrim™ Enzyme Formula promotes efficient disintegration and dissolution of the formula ingredients to ensure optimal benefits—proprietary weight management formula.
   Gymnema sylvestre—promotes optimal insulin function and helps maintain glucose levels within the normal range.
   Green Tea Leaf Extract—EGCG and caffeine
   TKN2 Thermogenic complex:
   Niacin—produces a mild ‘histamine’ flush while its vasodilating properties enhance blood and nutrient distribution to tissues. Also exerts a minor thermogenic effect.

### Kola Nut

### L-Taurine

### SEP711C3G is Designed to:

1. Improve the efficiency of energy metabolism and fat burning
2. Improve tolerance to stress (reduce the impact of stress on the body)
3. Promote learning, memory, cognition, healthy brain function, and longevity (anti-aging)
4. Support a happier mood
5. Promote healthy cravings
6. Reduce the time needed for and improve the quality of satiety or the satisfaction from pleasurable experiences (like eating)
7. Improve brain, nerve and glandular (neuro-endocrine) function
8. Promote competent immune function
9. Promote healthy blood sugar and blood lipid levels within the normal range
10. Promote healthy fat loss and weight management!

### Benefits and Features:

The Neuroadaptagen Amino-Acid Therapy formulas [NAAT™] of the invention provide a unique combination of diet ingredients, including thermogenic and energy supporting ingredients that will help one lose weight and increase lean muscle.

In terms of craving behavior each neutraceuitical developed will address a specific brain dysfunction. In this regard, NAAT™ has been designed to significantly reduce carbohydrate bingeing. The mechanism for this effect involves the pharmacological principal-like treats like. In this case, the common release of dopamine at the reward site by glucose is linked to aberrant glucose seeking behavior. This compulsive drive for dopamine is affected by the use of the patented ('724 patent) Synaptose™ composition which works on the brain reward system to mimic the action of glucose on nucleus accumbens neurons to release dopamine. Dopamine when released activates dopamine D₂ receptors. When these receptors are activated by dopamine the system is driven to attain pleasure and well-being. In general, since deficits have been found in brain chemical functions underlying craving behavior, and since these deficits may be alleviated by facilitated dopamine release consequent to the use of substances such as glucose, combining amino-acid precursors and enkephalinase inhibition may stimulate the brain's reward system and compensate for neurotransmitter imbalance (thereby attenuating glucose craving behavior). In an attempt to understand that carbohydrate seeking behavior, is a subset of generalized craving behavior (RDS) due in part to low dopamine function (an impaired reward cascade), scientists believe individuals self-heal through biochemical attempts to alleviate hypodopaminergic activity via glucose-reward site interaction. Since the brain is made up of 200 billion cells and these cells require good nutrition, which includes minerals, vitamins, trace metals and amino acids, NAAT™ is a special blend with brain stabilizing and metabolic properties. It is noteworthy, that since it is known that dopamine D₂ occupancy by dopamine D₂ agonists increase D₂ receptors, it is the contention that the use of this product would induce a constant release of dopamine, which will occupy dopamine D₂ receptors, and ultimately reduce craving behavior due to a genetic deficiency of carrying the Dopamine D₂ Receptor A1 allele (expression of low D₂ receptor number).

### EXAMPLES

### Imaging Studies: Genes and Weight Gain

Acute oral NAAT™ on reward circuitry during uprotracted abstinence following psychostimulant dependence was tested in ten subjects associated with G & G Holistic Addiction Treatment Center of North Miami Beach, Fla. These subjects were diagnosed as having severe psychostimulant dependence and have been in recovery for at least two years.

As part of the inclusion criteria, each patient was urine tested to determine the absence or presence of any psychoactive drug (illicit). None of the subjects tested showed a positive, urine-based drug test. Therefore, they were subsequently admitted to the study.

To date in preliminary analysis it was found that a comparison of the FFT absolute Power (uVSq) of alpha (8-12 Hz) demonstrated higher activity in the NAAT™ group compared to the placebo group. Similarly, observing the FFT absolute Power (uVSq) of low beta (12.0 15 hz), the activity was considerably larger in the NAAT™ group compared to the placebo group Finally, there was a consistent effect of NAAT™ on frontal regions when compared to placebo. The p values for group 1 (NAAT™) versus Group 2 (Placebo) for a between-group analysis of week 1 and week 2 whereby group comparisons utilizing T-tests were performed resulted in significant differences.

Imaging studies were also performed in an attempt to establish a measurable magnitude of effect and mechanism of action. The results of initial qEEG studies show an interaction of NAAT and meso-limbic activation leading to “normalization” of abnormal dopaminergic function in anticipation of patients carrying a number of reward gene polymorphisms.

NAAT™ appears to be a D2 natural non-addicting agonist. Further fMRI and PET scan analysis will be conducted to determine chronic induction of D2/D3 receptors, especially in DRD2 A1 allele carriers and direct interaction at D2 receptor NAc interaction. NAAT™ appears to “normalize” brain abnormalities associated with drug dependence (alcohol, heroin, and psycho stimulants) induced by dopaminergic deficiency by acting as a Dopaminergic receptor agonists during protracted abstinence in polydrug abusers. This mechanism is supported by other studies showing enhanced treatment response in only A1 vs. A2 carriers. The greatest effect is expected to occur in those individuals possessing the DRD2 TAq A1 allele. It is anticipated that long-term activation of dopaminergic receptors (i.e., DRD2 receptors) will up-regulate D2 receptor expression, leading to enhanced dopamine sensitivity and an increased sense of happiness.

### Nutrigenomics of Obesity Examples

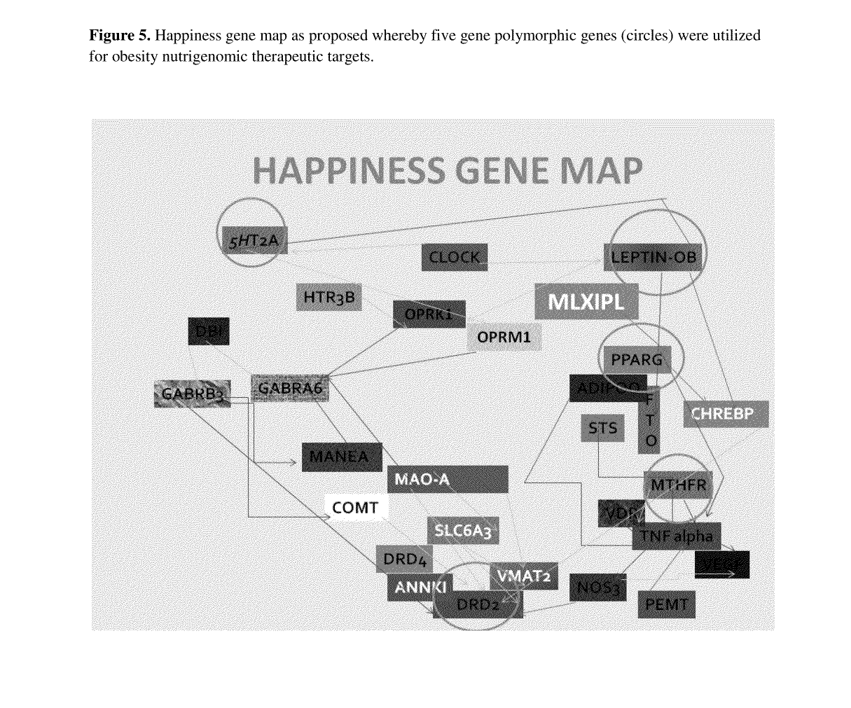

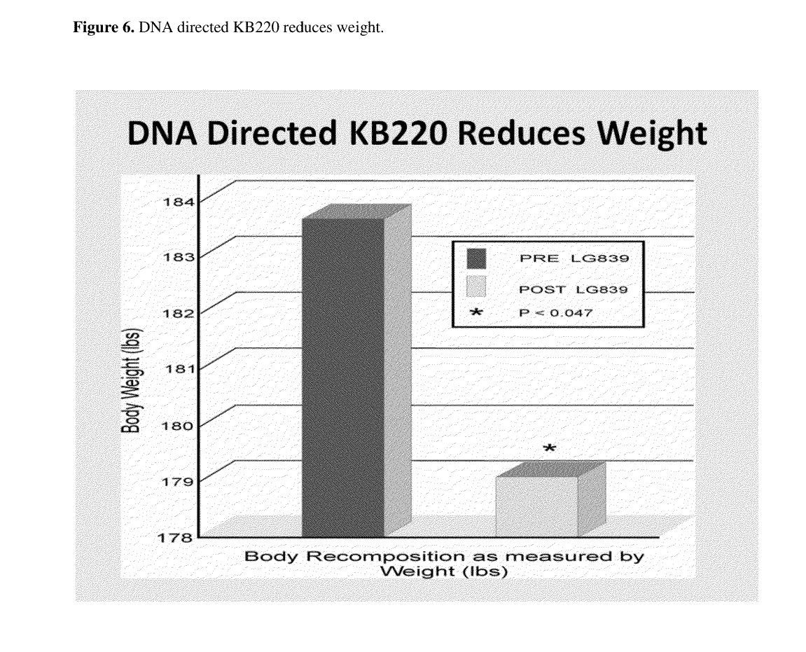

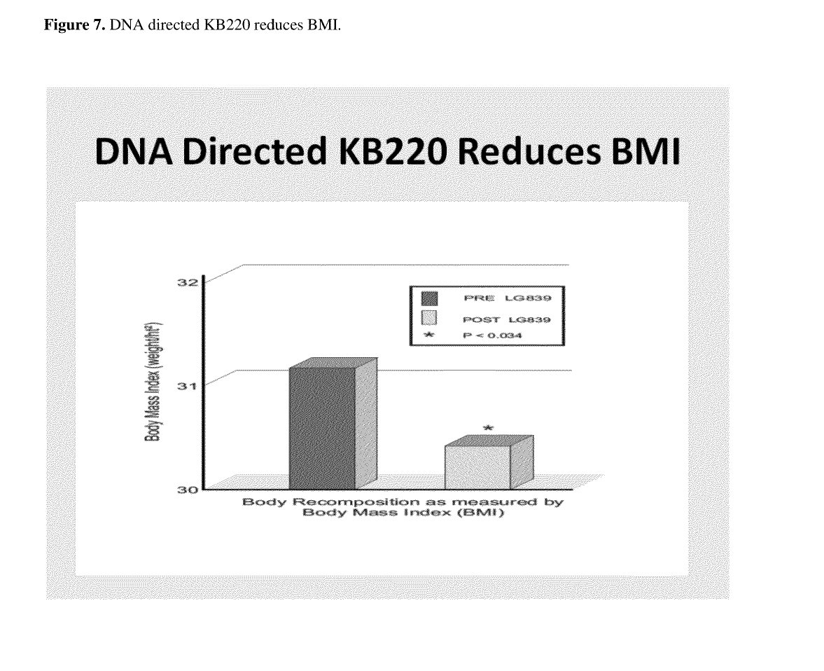

Following on inventor Blum's U.S. Pat. No. 6,955,873, nutrigenomic principles have been utilized to target certain gene polymorphisms, including, but not limited to, 5HT2a receptors, PPAR-Gamma, MTHFMR, LEP-OB, and DRD2 genes (FIG. 5) with significant reductions in both weight (see FIG. 6) and BMI (see FIG. 7). In these studies it was also found that there was 2-fold better compliance with carriers of the DRD2 A1 allele compared to carriers of the DRD2 A2 allele.

The first study assessed systematically the weight management effects of a novel experimental DNA-customized nutraceutical, KB220 variant. A total of 1058 subjects who participated in the overall D.I.E.T. study were genotyped and administered anKB220 variant based on polymorphic outcomes. A subset of 27 self-identified obese subjects of Dutch descent, having the same DNA pattern of four out of the five candidate genes tested (chi-square analysis) as the entire data set, was subsequently evaluated. Simple t tests comparing a number of weight management parameters before and after 80 days of treatment with KB220 variant were performed. Significant results were observed for weight loss, sugar craving reduction, appetite suppression, snack reduction, reduction of late night eating (all P<0.01), increased perception of overeating, enhanced quality of sleep, increased happiness (all P<0.05), and increased energy (P<0.001). Polymorphic correlates were obtained for a number of genes (LEP, PPAR-γ2, MTHFR, 5-HT2A, and DRD2 genes) with positive clinical parameters tested in this study. Of all the outcomes and gene polymorphisms, only the DRD2 gene polymorphism (A1 allele) had a significant Pearson correlation with days on treatment (r=0.42, P=0.045).

The second study tested the hypothesis that genotyping certain known candidate genes would provide DNA-individualized customized nutraceuticals that may have significant influence on body re-composition by countering various genetic traits. It is well known that obesity and related symptoms significantly aggravates type-2 diabetes. Both obesity and diabetes are influenced by the interaction of both genes and environmental factors. Exploration of the current literature hasidentified a number of candidate genes to be associated with both of these two disorders and include amongst others the dopamine D2 receptor (DRD2), methylenetetrahydrofolate reductase (MTHFR), serotonin receptor (5-HT2a), Peroxisome Proliferator-Activated Receptor gamma (PPAR-γ), and Leptin (OB) genes. In the second study, the impact of polymorphisms of these five candidate genes was systematically evaluated as important targets for the development of a DNA-customized nutraceutical KB220 [dl phenylalanine, chromium, 1-tyrosine other select amino-acids and adaptogens]) to combat obesity with special emphasis on body recomposition as measured by Body Mass Index (BMI). A total of 21 individuals were evaluated in a preliminary investigational study of LG839. Based on the results of buccal swab genotyping of each subject, an individualized customized nutraceutical formula was provided as a function of measured gene polymorphisms of the five gene candidates assessed. At the inception of the study and every two weeks subsequently, each subject completed a modified Blum-Downs OPAQuE Scale™ [Overweight Patient Assessment Questionnaire] The alleles included the DRD2 A1; MTHFR C 677T; 5HT2a 1438G/A; PPAR-γPro12A1a and Leptin Ob1875<208 bp. Pre- and post ad hoc analysis revealed a significant difference between the starting BMI and the BMI following an average of 41 days (28-70d) of KB220 variant intake in the 21 individuals. The pre-BMI was 31.2 (weight/Ht2) compared to the post BMI of 30.4 (weight/Ht2) with a significance value of P<0.034 (one tailed). Similarly the pre-weight in pounds (lb) was 183.52 compared to the post weight of 179 lb with a significance value of P<(0.047). We also found trends for reduction of late night snacking, carbohydrate craving reduction, reduction of stress, reduction of waist circumference. Moreover, in the 41-day period a trend in weight loss was found whereby 71.4% of subjects lost weight. Thus 15 out of 21 subjects lost weight with a z score of 2.4 and significance value of P<(0.02). In this group 53% lost on average over 2.5% of their starting weight.

### RDS Gene Map

### An RDS Gene Map to Assist in Nutrigenomic Solutions for Obesity and Eating Disorders

RDS is linked to flawed dopamine metabolism, and especially to low D2 receptor density. Moreover, RDS results from a dysfunction in the mesolimbic system of the brain, which directly links abnormal craving behavior with a defect in the Dopamine D2 Receptor Gene (DRD2) as well as other dopaminergic genes (D1, D3, D4, and D5, DATA1, MAO, COMT), including many genes associated with the brain reward function, as listed in Table 2, below.

TABLE 2
Genes associated with RDS

| REWARD-DEPENDENCE- | |
|---|---|
| PATHWAY | CANDIDATE GENES |
| Signal Transduction | ADCY7 |
| Signal Transduction | AVPR1A |
| Signal Transduction | AVPR1B |
| Signal Transduction | CDK5R1 |
| Signal Transduction | CREB1 |
| Signal Transduction | CSNKLE |
| Signal Transduction | FEV |
| Signal Transduction | FDS |
| Signal Transduction | FOSL1 |
| Signal Transduction | FOSL2 |
| Signal Transduction | GSK3B |
| Signal Transduction | JUN |
| Signal Transduction | MAPK1 |
| Signal Transduction | MAPK3 |
| Signal Transduction | MAPK14 |
| Signal Transduction | MPD2 |
| Signal Transduction | MGFB |
| Signal Transduction | NTRK2 |
| Signal Transduction | NTSR1 |
| Signal Transduction | NTSR2 |
| Signal Transduction | PPP1R1B |
| Signal Transduction | PRKCE |
| Serotonin | HTRIA |
| Serotonin | HTRIB |
| Serotonin | HTR2A |
| Serotonin | HTR2C |
| Serotonin | HTR3A |
| Serotonin | HTR3B |
| Serotonin | MAOA |
| Serotonin | MAOB |
| Serotonin | SLC64A |
| Serotonin | TPH1 |
| Serotonin | TPH2 |
| Opioid | OPRMI |
| Opioid | OPRKI |
| Opioid | PDYN |
| Opioid | PMOC |
| Opioid | PRD1 |
| Opioid | OPRL1 |
| Opioid | PENK |
| Opioid | PNOC |
| GABA | GABRA2 |
| GABA | GABRA3 |
| GABA | GABRA4 |
| GABA | GABRA6 |
| GABA | GABRB1 |
| GABA | GABRB2 |
| GABA | GABRB3 |
| GABA | GABRD |
| GABA | GABRE |
| GABA | GABRG2 |
| GABA | GABRG3 |
| GABA | GABRQ |
| GABA | SLC6A7 |
| GABA | SL6A11 |
| GABA | SLC32A1 |
| GABA | GAD1 |
| GABA | GAD2 |
| GABA | DB1 |
| Dopamine | COMT |
| Dopamine | DDC |
| Dopamine | DRD1 |
| Dopamine | DRD2 |
| Dopamine | DRD3 |
| Dopamine | DRD4 |
| Dopamine | DRD5 |
| Dopamine | SLC18A2 |
| Dopamine | SLC6A3 |
| Dopamine | TH |
| Cannabinoid | CNR1 |
| Cannabinoid | FAAH |
| Cholinergic | CHRMI |
| Cholinergic | CHRM2 |
| Cholinergic | CHRM3 |
| Cholinergic | CHRM5 |
| Cholinergic | CHRNA4 |
| Cholinergic | CHRNB2 |
| Adrenergic | ADRA1A |
| Adrenergic | ADRA2B |
| Adrenergic | ADRB2 |
| Adrenergic | SLC6A2 |
| Adrenergic | DRA2A |
| Adrenergic | DRA2C |
| Adrenergic | ARRB2 |
| Adrenergic | DBH |
| Glycine | GLRA1 |
| Glycine | GLRA2 |
| Glycine | GLRB |
| Glycine | GPHN |
| NDMA | GR1K1 |
| NDMA | GRINI |
| NDMA | GRIN2A |
| NDMA | GRIN2B |
| NDMA | GRIN2C |
| NDMA | GRM1 |
| Stress | CRH |
| Stress | CRHEP |
| Stress | CRHR1 |
| Stress | CRHR2 |
| Stress | GAL |
| Stress | NPY |
| Stress | NPY1R |
| Stress | NPY2R |
| Stress | NPY5R |
| Drug Metabolizing | ALDH1 |
| Drug Metabolizing | ALDH2 |
| Drug Metabolizing | CAT |
| Drug Metabolizing | CYPZE1 |
| Drug Metabolizing | ADH1A |
| Drug Metabolizing | ADH1B |
| Drug Metabolizing | ADH1C |
| Drug Metabolizing | ADH4 |
| Drug Metabolizing | ADH5 |
| Drug Metabolizing | ADH6 |
| Drug Metabolizing | ADH6 |
| Drug Metabolizing | ADH7 |
| Others | BDNF |
| Others | CART |
| Others | CCK |
| Others | CCKAR |
| Others | CLOCK |
| Others | HCRT |
| Others | LEP |
| Others | NR3C1 |
| Others | SLC29A1 |
| Others | TAC |

The genesis of all behavior, be it “normal” (socially acceptable) or “abnormal” (socially unacceptable), derives from an individual's genetic makeup at birth. This genetic predisposition, due to multiple gene combinations and polymorphisms, is expressed differently based on numerous environmental factors including family, friends, educational and socioeconomic status, environmental contaminant exposure, and the availability of psychoactive drugs, including food. The core of predisposition to these behaviors is a set of genes interacting with the environment, which promote a feeling of wellbeing via neurotransmitter interaction at the “reward center” of the brain (located in the meso-limbic system), leading to normal dopamine release.

Subjects afflicted with RDS carry polymorphic genes in dopaminergic pathways that result in hypo-dopominergic function caused by a reduced number of dopamine D2 receptors, reduced synthesis of dopamine (by dopamine beta-hydroxylase), reduced net release of pre-synaptic dopamine (from, e.g., the dopamine D1 receptor), increased synaptic clearance due to a high number of dopamine transporter sites (dopamine transporter), and low D2 receptor densities (dopamine D2 receptor), making such people more vulnerable to addictive behaviors. The RDS concept involves shared genes and their mRNA expression and behavioral tendencies, including dependence on alcohol, psycho-stimulants, marijuana, nicotine (smoking), and opiates, altered opiate receptor function, carbohydrate issues (e.g., sugar-binging), obesity, pathological gambling, sex addiction, premeditated aggression, stress, pathological aggression, and certain personality disorders, including novelty-seeking and sex addiction. The common theme across all of these substances and behaviors is that they induce pre-synaptic dopamine release. Spectrum disorders such as ADHD, Tourettes Syndrome, and Autismare also included due to dopamine dysregulation. As well as other rare mutations (have been associated with Tourettes and Autism. One example includes the association with Neuroligin 4 (NLGN4) is a member of a cell adhesion protein family that appears to play a role in the maturation and function of neuronal synapses.

It is well know that stress induces the preferential release of the circulatory hormone cortisol in humans. It is well know that lipolysis is the major activity that is involved in the burning of fat in adipose tissue. Ottosson et. al. clearly showed that cortisol significantly reduced the basal rate of lipolysis (p<0.01) and the catecholamine lipoysis stimulators isoprenaline and noradreanline in vitro. Thus, cortisol will increase rather than decrease fat burning. In addition, the patogenesis of obesity has been suggested to be intimately linked to the catechoalminegic regulation of lipolysis and the function of the sympathetic nervous system. Norepinephrine and epinephrine activate lipolysis via B₁ and B₂ and B₃-adrenoreceptors and inhibit it via alpha₂-adrenoreceptors, and these neurotransmitters are the most important lipolytic substances on vivo. Defects of the catecholamine-induced lipolysis have been observed in a number of obese subjects, and polymorphisms of the B2- and B 3 receptors.

By adding both Passiflora and KB220BZ a combination heretofore never combined we propose a synergistic effect on stress production and enhanced catecholamine synthesis. We further believe that these ingredients coupled together would induce a reduction of plasma cortisol on humans. This will indeed then enhance lipolysis and increase fat burning.

In essence, this novel formulation (see table 1, above, for preferred ingredients) will promote the synthesis of the brain reward neurotransmitters like serotonin and catecholamines and through its effect on the natural opioids will by virtue of inhibiting GABA cause a significant release of dopamine at the nucleus accumbens. The proposed list of ingredients will reduce inflammation one cause of reduced insulin sensitivity leading to diabetes and obesity. This constant release of possibly therapeutic amounts of dopamine (anti-stress substance) occupies dopamine D₂ receptors, especially in carriers of the A₁ allele (low D₂ receptors and high glucose craving), and over time (possibly 6-8 weeks) effects RNA transcription leading to a proliferation of D₂ receptors, thereby, reducing craving for carbohydrates. Evidence for anorectic actions of dopaminergic stimulators like Amphetamines I (ephedra) have been found to work via activation of both D1 ands D2 dopamine receptors. In addition, elucidation of the composition, characteristics and properties of stabilized (−) HCA compounds of GcEs is essential to differentiate effective sources from ineffective and substantiate the actual active ingredients in such mineral-based complexes. Recent research demonstrates intake of 4500 mg/d of a novel IH464 GcE containing 720 mg of K and 495 mg of Ca bound by (−) HCA for 8 weeks, while consuming a 2000 Kcal/d diet, produced safe and effective loss of body fat and improved BMI without stimulating the central nervous system. Other ingredients as listed in the example will also provide important benefits such as anti-craving, anti-stress, enhancement of serotonin, energy and metabolism induction, appetite suppression, starch blocking, glucose stabilization, fat burning, and general nutrition as well as neurotransmitter rebalancing. The increase in dopamine function for example will influence executive functioning important in making appropriate judgment to eat or not to eat. This is further influenced by adding phospholipid-based ingredients. Collateral benefits of lowered food intake and improved serotonin, insulin, lipid and leptin metabolism provide valuable evidence that this compound addresses multiple pathways in achieving sustainable healthy fat loss and improvements in body mass index while averting the consequences of rapid “weight loss” (a term that falsely represents the real culprit which is fat gain or unwanted storage-energy conservation) induced by CNS stimulation and/or calorie deprivation.

Importantly, each listed ingredient in this application is exclusive to the inventors when combined with Synaptose or any KB220 variant with or without a glycoside.

Through a series of both neurogenetic and clinical experiments it is becoming increasingly clear that this novel formulation is the first neuroadaptagen known to activate the brain reward circuitry. Ongoing research repeatedly confirms the numerous clinical effects ultimately result in significant benefits for victims having genetic antecedents for all addictive, compulsive and impulsive behaviors. These behaviors are all correctly classified under the rubric of “Reward Deficiency Syndrome” (RDS). Preliminary findings in United States using qEGG and China using fMRI regarding the effects of oral NAAT™ in addicts on activation of brain reward circuitry provides potentially exciting results. It seems from this preliminary data, utilizing an fMRI 2×2 design at resting state, NAAT™ in comparison to placebo shows activation of the caudate brain region and potentially a smoothing out of heroin induced putamen abnormal connectivity.

The invention represents a paradigm shift in understanding that obesity is a subtype of RDS behaviors that originates in the brain. Various benefits of the invention include the following:

### Stress Reduction

### Enhancement of Sleep

### Increased Energy Levels

### Generalized Well-Being

Craving Behavior Reduction (sweets/carbs)

### Mental Focus/Memory

### Blood Sugar Levels

### Food Consumption Reduction

### Loss Of Inches

### Loss Of Fat

### Blood Pressure Reduction

### Improvement of Workout Performance

Reduction of drug seeking behavior (alcohol, nicotine, cocaine, marijuana, opiates, etc)

### Reduction of Hyperactivity

Reduction of Cholesterol

What is claimed:

1. A composition comprising an effective amount of:
(a) at least one substance that inhibits the enzymatic destruction of an opioid (opiate) neuropeptide (preferably an amino acid, peptide, or structural analogue or derivative thereof);
(b) a neurotransmitter synthesis-promoting amount of at least one neurotransmitter precursor, preferably a dopamine precursor such as L-Tyr, L-Phe, or L-Dopa, a serotonin precursor (e.g., L-Trp, and 5-hydroxytryptophan), and/or a gamma amino butyric acid (GABA) precursor, for example, L-glutamine, 1-glutamic acid, and L-glutamate (or GABA itself);
(c) a tryptophan concentration-enhancing amount of any chromium salt (for example chromium picolinate or chromium nicotinate);
(d) a neurotransmitter synthesis-promoting amount of at least one neurotransmitter synthesis promoting substance or catabolic inhibitor selected from the group consisting of rhodiola and huperzine; and
(e) at least one of the following:
(e)(1) Rhodiola;
(e)(2) Passionflower (incarnata);
(e)(3) vitamin B6;
(e)(4) vitamin B 1;
(e)(5) carnitine;
(e)(6) Rhododendron;
(e)(7) (−)-Mineral salts of (−)Hyroxycitric acid;
(e)(8) Gymnema sylvestre; and
(e)(9) calcium.

2. A composition according to claim 1, comprising:

| | Desired Thera- | | |
|---|---|---|---|
| Ingredient | peutic Dose | | Dosage Range |
| DL-Phenylalanine | 2170 | mg | 10 mg to 10.000 mg |
| L-tyrptophan | | | 1 mg to 2000 mg |
| 5-Hydroxytryptaphan | 50 | mg | 1 mg to 500 mg |
| L-Glutamine | 50 | mg | 1 mg to 500 mg |
| L-Tyrosine | 775 | mg | 1 mg to 5000 mg |
| Chromium Polynicotinate | 4 mg (400 mcg | | 10 mcg to 100 mg |
| | elemental Cr) | | |
| Rhodiola | 170 | mg | 5 mg to 200 mg |
| Passion Flower | 300 | mg | 1 mg to 3000 mg |
| (incarnata) | | | |
| Vitamin B6 | 20 | mg | 10 mcg to 500 mg |
| Pyridoxine HCl (10) | | | |
| Pyridoxal 5-Phosphate (10) | | | |
| Vitamin B1 (Thiamine) | 31 | mg | 10 mcg to 1000 mg |
| Carnitine | 250 | mg | 1 mg to 5000 mg |
| Rhododendron | 100-200 | | 1 mg to 5000 mg |
| (−)- Mineral salts | 4500 | mg | 10 mg to 10,000 mg |
| of (−)Hyroxycitric | | | |
| acid (HCAMin) | | | |
| Gymnema sylvestre | 25 | mg | 1 mg to 500 mg |
| Calcium | 1200 | mg | 1-3000 mg |
| CogniTrim ™ | (combination of | | |
| | SH1028 &NOPE2G2) | | |
| CurQFen ™ | 100 | mg | 1 mg to 2000 mg |
| PhosphoLean ™ | 100 | mg | 1 mg to 5000 mg |
| SH1028 Choline | 275 | mg | 1 mg to 5000 mg |
| alphoscerate | | | |
| GlucodOX ™ | 70 | mg | 1 mg to 3000 mg |
| Irvingia gabonensis | 300 | mg | 1 mg to 5000 mg |
| seed extract (OB131) | | | |
| Huperzine A | 20 | mg | 100 mcg to 1000 mg |
| (1% concentration) | | | |
| Bauhinia variegata | 100 | mg | 1 mg to 2000 mg |
| extract | | | |
| Niacin | 30 | mg | 1-100 mg |
| Taurine | 500 | mg | 1 mg to 3000 mg |
| Kola Nut | 300 | mg | 1 mg to 3000 mg |
| Zehntose | 200 | mg | 1 mg to 5000 mg |
| Metallosaccharides | | | |
| complex | | | |
| Green Tea extract | 100 | mg | 1 mg to 2000 mg |
| Weight Management | 100 | mg | 1 mg to 5000 mg |
| Enzyme Complex | | | |

3. A method for treating or preventing obesity, comprising administering to a patient suffering or recovering from or predisposed to develop obesity an amount of composition according to claim 1 effective to treat or prevent obesity.

4. A method according to claim 1, further comprising before administration of said composition performing an analysis of the patient with respect to one or more genes associated with Reward Deficiency Syndrome.

5. A method according to claim 4, wherein one or more of the genes associated with Reward Deficiency Syndrome are selected from the group of consisting of those genes listed in Table 2.
